(19) United States
(12) Patent Application Publication
(10) Pub. No.: US 2011/0104290 A1
(43) Pub. Date: May 5, 2011

(54) COMPOSITIONS FOR REDUCING BLOOD GLUCOSE LEVEL AND USES THEREOF

(76) Inventor: Sicheng Chen, Sichuan (CN)

(21) Appl. No.: 12/997,824

(22) PCT Filed: Jun. 12, 2008
(86) PCT No.: PCT/CN2008/001141

§ 371 Date: Dec. 13, 2010

(51) Int. Cl. A61K 9/14; A61K 36/906; A61P 3/10

(52) U.S. Cl. 424/489; 424/728

(57) ABSTRACT

A composition, its preparation and use for reducing the blood glucose level in a subject, comprising Radix Ginseng, Rhizoma Atractylodis Macrocephalae, Radix Glycyrrhizae, Ginger, and one or more components selected from the group consisting of Tartarian Buckwheat, Rhizoma Phragmitis (Phragmites communis), Radix Trichosanthis, and Folium Mori.

### FIELD OF THE INVENTION

The present invention relates in general to nutraceutical and pharmaceutical compositions. More specifically, the invention provides compositions for treating diabetes.

### BACKGROUND OF THE INVENTION

Diabetes is a disease in which the body does not produce or properly use insulin. Insulin is a hormone that is needed to convert sugar, starches, and other food into energy needed for daily life. Patients with diabetes typically have higher blood glucose levels. The cause of diabetes continues to be a mystery, although both genetics and environmental factors such as obesity and lack of exercise appear to play roles. Effective treatments are needed.

### SUMMARY OF THE INVENTION

This invention is based, at least in part, upon the unexpected discovery that compositions can be prepared from several plants for reducing blood glucose levels in subjects.

Accordingly, in one aspect, the invention features a composition comprising Radix Ginseng, Rhizoma Atractylodis Macrocephalae, Radix Glycyrrhizae, Ginger, and one or more components selected from the group consisting of Tartarian Buckwheat, Rhizoma Phragmitis (Phragmites communis), Radix Trichosanthis, and Folium Mori.

The invention also features a composition comprising Radix Ginseng, Rhizoma Atractylodis Macrocephalae, Radix Glycyrrhizae, Ginger, and one or more components selected from the group consisting of Tartarian Buckwheat, Rhizoma Phragmitis (Phragmites communis), Radix Trichosanthis, and Folium Mori, wherein the composition does not consist of Radix Ginseng, Rhizoma Atractylodis Macrocephalae, Radix Glycyrrhizae, Ginger, Tartarian Buckwheat, and Radix Trichosanthis.

In another aspect, the invention features a method of preparing a composition. One method comprises admixing Radix Ginseng, Rhizoma Atractylodis Macrocephalae, Radix Glycyrrhizae, Ginger, and one or more components selected from the group consisting of Tartarian Buckwheat, Rhizoma Phragmitis (Phragmites communis), Radix Trichosanthis, and Folium Mori to form a composition. Another method comprises admixing Radix Ginseng, Rhizoma Atractylodis Macrocephalae, Radix Glycyrrhizae, Ginger, and one or more components selected from the group consisting of Tartarian Buckwheat, Rhizoma Phragmitis (Phragmites communis), Radix Trichosanthis, and Folium Mori to form a composition, wherein the composition does not consist of Radix Ginseng, Rhizoma Atractylodis Macrocephalae, Radix Glycyrrhizae, Ginger, Tartarian Buckwheat, and Radix Trichosanthis.

In some embodiments, the amount of Radix Ginseng is in the range of 1-50% by weight of a composition of the invention, the amount of Rhizoma Atractylodis Macrocephalae is in the range of 5-50% by weight of the composition, the amount of Radix Glycyrrhizae is in the range of 1-20% by weight of the composition, and the amount of Ginger is in the range of 5-30% by weight of the composition.

In some embodiments, the total amount of Radix Ginseng, Rhizoma Atractylodis Macrocephalae, Radix Glycyrrhizae, and Ginger is in the range of 30-90% by weight of a composition of the invention, and the total amount of the one or more components selected from the group consisting of Tartarian Buckwheat, Rhizoma Phragmitis (Phragmites communis), Radix Trichosanthis, and Folium Mori is in the range of 10-70% by weight of the composition.

In some embodiments, Radix Ginseng, Rhizoma Atractylodis Macrocephalae, Radix Glycyrrhizae, Ginger, Tartarian Buckwheat, Rhizoma Phragmitis (Phragmites communis), Radix Trichosanthis, or Folium Mori is in the form of superfine powder or extract powder.

For example, the size of the superfine powder may be greater than 300 mesh.

The extract may be prepared by extracting slices of Radix Ginseng,

Rhizoma Atractylodis Macrocephalae, Radix Glycyrrhizae, Ginger, Tartarian Buckwheat, Rhizoma Phragmitis (Phragmites communis), Radix Trichosanthis, or Folium Mori with 40-85% by volume ethanol in water. The method may further comprise steps of allowing ethanol to evaporate, absorbing the remaining aqueous solution to large-pore absorbent resins, separating the extract from the resins (e.g., by desorption of resin with 60-80% by volume ethanol in water and removing the resins), and collecting the extract (e.g., allowing ethanol to evaporate and spraying-dry the extract to form powder).

In some embodiments, a half amount of a composition of the invention is in the form of superfine powder and the other half of the composition is in the form of extract powder.

In some embodiments, a composition of the invention is in the form of tablet or capsule. The tablet may weigh 1.5 g; the total amount of Radix Ginseng, Rhizoma Atractylodis Macrocephalae, Radix Glycyrrhizae, Ginger, and the one or more components selected from the group consisting of Tartarian Buckwheat, Rhizoma Phragmitis (Phragmites communis), Radix Trichosanthis, and Folium Mori in the tablet may be 1.2 g. In some embodiments, a composition of the invention comprises Radix Ginseng, Radix Glycyrrhizae, Ginger, Rhizoma Atractylodis Macrocephalae, Tartarian Buckwheat, Radix Trichosanthis, and Rhizoma Phragmitis (Phragmites communis); Radix Glycyrrhizae, Ginger, Rhizoma Atractylodis Macrocephalae, Tartarian Buckwheat, Radix Ginseng, and Rhizoma Phragmitis (Phragmites communis); Radix Ginseng, Radix Glycyrrhizae, Ginger, Radix Trichosanthis, and Rhizoma Atractylodis Macrocephalae; Radix Ginseng, Rhizoma Atractylodis Macrocephalae, Radix Glycyrrhizae, Ginger, Radix Trichosanthis, and Folium Mori; or Radix Ginseng, Radix Glycyrrhizae, Ginger, Rhizoma Atractylodis Macrocephalae, Rhizoma Phragmitis (Phragmites communis), and Folium Mori.

A composition prepared according to any of the methods described above is within the invention.

Furthermore, the invention provides a method of reducing the blood glucose level in a subject such as a human. The method comprises administering to a subject in need thereof (e.g., a subject suffering from diabetes) an effective amount of a composition of the invention. The composition may be administered orally or intragastrically. As an example, a composition of the invention in the form of 1.5 g tablet may be administered to a human subject suffering from diabetes at a daily dose of 10-15 tablets.

The above-mentioned and other features of this invention and the manner of obtaining and using them will become more apparent, and will be best understood, by reference to the following description drawing.

### BRIEF DESCRIPTION OF THE FIGURE

FIG. 1 demonstrates the effect of Composition 1 on blood glucose levels in rats.

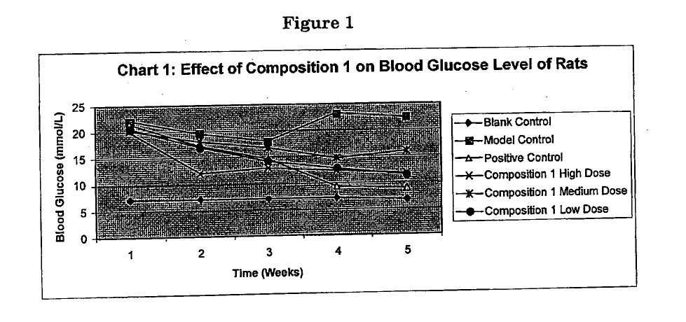

### DETAILED DESCRIPTION OF THE INVENTION

One object of the present invention is to provide a composition for reducing blood glucose levels in subjects. The composition is prepared from various plants grown worldwide, and preferably, in China.

More specifically, a composition of the invention comprises Radix Ginseng, Rhizoma Atractylodis Macrocephalae, Radix Glycyrrhizae, Ginger, and one or more components selected from the group consisting of Tartarian Buckwheat, Rhizoma Phragmitis (Phragmites communis), Radix Trichosanthis, and Folium Mori. Another composition of the invention comprises Radix Ginseng, Rhizoma Atractylodis Macrocephalae, Radix Glycyrrhizae, Ginger, and one or more components selected from the group consisting of Tartarian Buckwheat, Rhizoma Phragmitis (Phragmites communis), Radix Trichosanthis, and Folium Mori, wherein the composition does not consist of Radix Ginseng, Rhizoma Atractylodis Macrocephalae, Radix Glycyrrhizae, Ginger, Tartarian Buckwheat, and Radix Trichosanthis.

Radix Ginseng is the root of Panax ginseng C.A. Mey., family Araliceae. It may also be from American ginseng.

Rhizoma Atractylodis Macrocephalae is the rhizome of Atractylodes macrocephala Koidz, family Compositae.

Radix Glycyrrhizae is the root of Glycyrrhiza uraleusis Fisch. or G. glabra L., family Leguminosae.

Ginger is the underground rhizome of the ginger plant, family Zingiberaceae.

Tartarian Buckwheat is a member of the Polygonaceae family.

Rhizoma Phragmitis (Phragmites communis) is the fresh or dried rhizome of Phragmites communis Trin. (Fam. gramineae).

Radix Trichosanthis is the dry root of Trichosanthis kirilowii maxim.

Folium Mori is the leaf of Morus alba L., family Moraceae.

In a composition of the invention, the amount of Radix Ginseng may be in the range of 1-50% (e.g., 1%, 5%, 10%, 20%, 30%, 40%, and 50%) (by weight) of the composition, the amount of Rhizoma Atractylodis Macrocephalae may be in the range of 5-50% (e.g., 5%, 10%, 20%, 30%, 40%, and 50%) (by weight) of the composition, the amount of Radix Glycyrrhizae may be in the range of 1-20% (e.g., 1%, 5%, 10%, 15%, and 20%) (by weight) of the composition, and the amount of Ginger may be in the range of 5-30% (e.g., 5%, 10%, 15%, 20%, 25%, and 30%) (by weight) of the composition.

In some embodiments of the invention, the total amount of Radix Ginseng, Rhizoma Atractylodis Macrocephalae, Radix Glycyrrhizae, and Ginger is in the range of 30-90% (e.g., 30%, 40%, 50%, 60%, 70%, 80%, and 90%) (by weight) of a composition of the invention, and the total amount of the one or more components selected from the group consisting of Tartarian Buckwheat, Rhizoma Phragmitis (Phragmites communis), Radix Trichosanthis, and Folium Mori is in the range of 10-70% (e.g., 10%, 20%, 30%, 40%, 50%, 60%, and 70%) (by weight) of the composition.

The components of a composition of the invention, Radix Ginseng, Rhizoma Atractylodis Macrocephalae, Radix Glycyrrhizae, Ginger, Tartarian Buckwheat, Rhizoma Phragmitis (Phragmites communis), Radix Trichosanthis, and Folium Mori may be in the form of superfine powder or extract powder.

By “superfine powder” is meant that the size of the powder is between 800 mesh and 300 mesh. For example, the size of the superfine powder may be greater than 300 mesh.

The extract may be prepared by extracting slices of Radix Ginseng, Rhizoma Atractylodis Macrocephalae, Radix Glycyrrhizae, Ginger, Tartarian Buckwheat, Rhizoma Phragmitis (Phragmites communis), Radix Trichosanthis, or Folium Mori with 40-85% (e.g., 40%, 50%, 60%, 70%, 80%, and 85%) ethanol in water. Ethanol may then be removed by evaporation, and the remaining aqueous solution absorbed by macroporous adsorbent resins having pores with a diameter between 100-400 Å. The extract may subsequently be desorbed from the resins, e.g., by desorbing the extract in 60-80% ethanol in water and removing the resins. The extract may then be collected, e.g., by allowing ethanol to evaporate and spraying-dry the extract to form powder.

A composition of the invention may be in the form of superfine powder or extract powder. Alternatively, a composition of the invention may be in the form of a mixture of superfine powder or extract powder. Preferably, a composition of the invention has half (by weight) of the composition in the form of superfine powder and the other half (by weight) in the form of extract powder.

Exemplary compositions of the invention include compositions containing a combination of Radix Ginseng, Radix Glycyrrhizae, Ginger, Rhizoma Atractylodis Macrocephalae, Tartarian Buckwheat, Radix Trichosanthis, and Rhizoma Phragmitis (Phragmites communis); a combination of Radix Glycyrrhizae, Ginger, Rhizoma Atractylodis Macrocephalae, Tartarian Buckwheat, Radix Ginseng, and Rhizoma Phragmitis (Phragmites communis); a combination of Radix Ginseng, Radix Glycyrrhizae, Ginger, Radix Trichosanthis, and Rhizoma Atractylodis Macrocephalae; a combination of Radix Ginseng, Rhizoma Atractylodis Macrocephalae, Radix Glycyrrhizae, Ginger, Radix Trichosanthis, and Folium Mori; and a combination of Radix Ginseng, Radix Glycyrrhizae, Ginger, Rhizoma Atractylodis Macrocephalae, Rhizoma Phragmitis (Phragmites communis), and Folium Mori.

A composition of the invention may be used for nutraceutical purposes, e.g., as a dietary supplement. A composition of the invention may also be admixed with a pharmaceutically acceptable carrier to form a pharmaceutical composition. “Pharmaceutically acceptable carriers” include solvents, dispersion media, coatings, antibacterial and antifungal agents, isotonic and absorption delaying agents, and the like, compatible with pharmaceutical administration.

A composition of the invention may be formulated to be compatible with its intended route of administration. (See, e.g., U.S. Pat. No. 6,756,196.) Examples of routes of administration include parenteral, e.g., intravenous, intradermal, subcutaneous, oral (e.g., inhalation), transdermal (topical), transmucosal, intragastric, and rectal administration. Solutions or suspensions used for parenteral, intradermal, intragastrical, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. The pH level can be adjusted with acids or bases, such as hydrochloric acid or sodium hydroxide. The parenteral preparation can be enclosed in ampoules, disposable syringes, or multiple dose vials made of glass or plastic.

In some embodiments, the compositions contain carriers that will protect the active ingredients against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. “Dosage unit form,” as used herein, refers to physically discrete units suited as unitary dosages for the subject to be treated, each unit containing a predetermined quantity of active ingredients calculated to produce the desired therapeutic effect in association with the required pharmaceutical carrier.

For example, a composition of the invention may be in the form of tablet or capsule for oral administration. In some embodiments, the tablet weighs 1.5 g. In some embodiments, the total amount of Radix Ginseng, Rhizoma Atractylodis Macrocephalae, Radix Glycyrrhizae, Ginger, and the one or more components selected from the group consisting of Tartarian Buckwheat, Rhizoma Phragmitis (Phragmites communis), Radix Trichosanthis, and Folium Mori in the tablet is 1.2 g.

Another object of the present invention is to provide a method of preparing a composition. One method involves admixing Radix Ginseng, Rhizoma Atractylodis Macrocephalae, Radix Glycyrrhizae, Ginger, and one or more components selected from the group consisting of Tartarian Buckwheat, Rhizoma Phragmitis (Phragmites communis), Radix Trichosanthis, and Folium Mori to form a composition. Another method involves admixing Radix Ginseng, Rhizoma Atractylodis Macrocephalae, Radix Glycyrrhizae, Ginger, and one or more components selected from the group consisting of Tartarian Buckwheat, Rhizoma Phragmitis (Phragmites communis), Radix Trichosanthis, and Folium Mori to form a composition, wherein the composition does not consist of Radix Ginseng, Rhizoma Atractylodis Macrocephalae, Radix Glycyrrhizae, Ginger, Tartarian Buckwheat, and Radix Trichosanthis.

Radix Ginseng, Rhizoma Atractylodis Macrocephalae, Radix Glycyrrhizae, Ginger, Tartarian Buckwheat, Rhizoma Phragmitis (Phragmites communis), Radix Trichosanthis, and Folium Mori are admixed according to the ratios and in the forms described above. For example, excipients that are the ingredients of a tablet other than the active ingredients may be added to granulate the powder mixture of plant components. Granulation is the process of collecting particles together by creating bonds between them. Examples of excipients include, but not limited to, magnesium stearate as glidant, silicon dioxide as antiadherent or lubricant. The granulated powder may then be tableted. Compositions so prepared are within the invention.

A composition of the invention can be used to reduce the blood glucose level in a subject, e.g., for treatment of diabetes. An effective amount of the composition is administered to a subject in need thereof via a proper route such as those described above.

As used herein, a “subject” refers to a human or animal, including all mammals such as primates (particularly higher primates), sheep, dog, rodents (e.g., mouse or rat), guinea pig, goat, pig, cat, rabbit, and cow. In a preferred embodiment, the subject is a human. In another embodiment, the subject is an experimental animal or animal suitable as a disease model.

A subject to be treated may be identified in the judgment of the subject or a health care professional, and can be subjective (e.g., opinion) or objective (e.g., measurable by a test or diagnostic method). For example, a subject to be treated may be a subject having an abnormally high blood glucose level, or a subject diagnosed with or suffering from diabetes.

A “treatment” is defined as administration of a substance to a subject with the purpose to cure, alleviate, relieve, remedy, prevent, or ameliorate a disorder, symptoms of the disorder, a disease state secondary to the disorder, or predisposition toward the disorder.

An “effective amount” is an amount of a composition that is capable of producing a medically desirable result in a treated subject. The medically desirable result may be objective (i.e., measurable by some test or marker) or subjective (i.e., subject gives an indication of or feels an effect).

In some embodiments, a composition of the invention is administered orally or intragastrically.

The dosage required for treating a subject depends on the choice of the route of administration, the nature of the formulation, the nature of the subject's illness, the subject's size, weight, surface area, age, and sex, other drugs being administered, and the judgment of the attending physician. Suitable dosages are typically in the range of 0.01-500.0 mg/kg. Wide variations in the needed dosage are to be expected in view of the different efficiencies of various routes of administration. For example, oral administration would be expected to require higher dosages than administration by intravenous injection. Variations in these dosage levels can be adjusted using standard empirical routines for optimization as is well understood in the art. Encapsulation of the active ingredients in a suitable delivery vehicle (e.g., polymeric microparticles or implantable devices) may increase the efficiency of delivery, particularly for oral delivery.

For example, a composition of the invention in the form of 1.5 g tablet described above may be administered to a human subject suffering from diabetes at a daily dose of 10-15 tablets.

A typical cycle of administration of a composition of the invention is from a week to a few months, e.g., two weeks, four weeks, two months, and four months. After the blood glucose level starts to drop in the subject, the dosage of the composition may be gradually reduced. The administration may terminate when the blood glucose level returns to normal.

For diabetes patients who take other medicines for diabetes, a composition of the invention may initially be taken concurrently with those medicines. After the blood glucose level starts to drop in the subject, the intake of the other medicines may be gradually reduced and eventually stopped. The subject may continue to take the composition of the invention, and if the blood glucose level continues to drop, the subject may gradually reduce the intake of the composition and eventually stop the intake of the composition when the blood glucose level returns to normal.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLES

### Example 1

### Preparation of Plant Superfine Powder

Plant was rinsed with clean water, dried in air, shattered, and further refined into superfine powders greater than 300 mesh.

### Preparation of Plant Extract Powder

Plant was rinsed with clean water, cut into slices, and extracted with 40-85% by volume ethanol in water. Ethanol was then removed by evaporation. The remaining aqueous solution was treated with macroporous adsorbent resins. The plant extract adsorbed by the resins was desorbed with ethanol (60-80% by volume). After removal of the resins, ethanol was evaporated off the plant extract. The plant extract was then spray-dried to form powder.

### Preparation of Dietary Supplement

The following plant superfine powders and plant extract powders were combined:

Radix Ginseng superfine powder 500 g,

Radix Glycyrrhizae superfine powder 200 g,

Ginger superfine powder 300 g,

Rhizoma Atractylodis Macrocephalae extract powder 300 g,

Tartarian Buckwheat extract powder 250 g,

Radix Trichosanthis extract powder 250 g,

Rhizoma Phragmitis extract powder 200 g.

Excipients were added to granulate the mixture. For example, magnesium stearate may be used as glidant, silicon dioxide may be used as antiadherent or lubricant. The granulated mixture was tableted. Each tablet weighed 1.5 g and contained 1.2 g plant superfine powders and plant extract powders.

The recommended daily amount for human consumption is 10-15 tablets.

### Example 2

The dietary supplement was prepared using the method described in Example 1 with the following material

Radix Glycyrrhizae superfine powder 300 g,

Ginger superfine powder 300 g,

Rhizoma Atractylodis Macrocephalae superfine powder 400 g,

Tartarian Buckwheat extract powder 200 g,

Radix Ginseng extract powder 300 g,

Rhizoma Phragmitis extract powder 500 g.

### Example 3

The dietary supplement was prepared using the method described in Example 1 with the following material

Radix Ginseng superfine powder 500 g,

Radix Glycyrrhizae superfine powder 100 g,

Ginger superfine powder 200 g,

Radix Trichosanthis extract powder 200 g,

Rhizoma Atractylodis Macrocephalae extract powder 300 g.

### Example 4

The dietary supplement was prepared using the method described in Example 1 with the following material

Radix Ginseng extract powder 500 g,

Rhizoma Atractylodis Macrocephalae extract powder 200 g,

Radix Glycyrrhizae extract powder 100 g,

Ginger extract powder 200 g,

Radix Trichosanthis superfine powder 400 g,

Folium Mori extract powder 600 g.

### Example 5

The dietary supplement was prepared using the method described in Example 1 with the following material

Radix Ginseng superfine powder 400 g,

Radix Glycyrrhizae superfine powder 100 g,

Ginger superfine powder 200 g,

Rhizoma Atractylodis Macrocephalae superfine powder 300 g,

Rhizoma Phragmitis extract powder 500 g,

Folium Mori extract powder 500 g.

### Example 6

### In Vivo Efficacy Study—Anti-Hyperglycemic Effects of Composition 1 Tablet in Streptozotocin (STZ)-Induced Hyperglycemic Rats

For the purpose of the present invention, the dietary supplement prepared in accordance with Example 1 is named Composition 1. Composition 1 is an herbal supplement used for control of blood glucose level. The purpose of this study was to assess the anti-hyperglycemic effect of Composition 1 tablet in STZ-induced hyperglycemic rats.

### Methods

### Establishment of STZ-Induced Hyperglycemic Rat Model

Sprague-Dawley (SD) rats weighing 230˜270 g were used to establish the hyperglycemic rat model. Animals were fasted for 18 hours before receiving a single intraperitoneal injection of 2% STZ at a dose of 40 mg/kg BW (2.0 ml/kg). Six SD rats were selected as the Blank Control Group and injected intraperitoneally with citric acid buffer at a dose of 2.0 ml/kg BW. All rats had free access to food and water after the injection. Blood samples were collected from rats' posterior orbits veins at 72 hours after the injection for determination of the blood glucose level. Successful establishment of hyperglycemic rat model was defined as a non-fasting venous blood glucose level≧13.8 mmol/L.

### Dose Design and Groups

The hyperglycemic rats were randomly divided into 6 groups, including Low Dose, Middle Dose, Secondary High Dose, and High Dose Composition 1 Groups, Metformin Control Group, and Model Control Group. A total of 7 experimental groups, including a Blank Control Group, were used in this study. The exact dosages were designed as follows:

The clinical dose of Composition 1 for human is 16.2 g/day. In this study, in accordance with the weight factor conversion between human and rat, Composition 1 tablets were administered daily to SPF-SD male rats at dose levels of 1.89, 2.7, 3.78, and 5.67 g/kg body weight/day for Low, Middle, Secondary High, and High Dose Groups, respectively.

The clinical dose of Metformin for human is 1˜1.5 g/day, which is about 0.025 g/kg body weight/day for an adult. The administration dose of Metformin for rat was calculated to be 0.25 g/kg body weight/day, 10 times of human clinical dose. The exact dose levels and groups are shown in Table 1.

TABLE 1
Dose Design and Groups

| | | | Times | | |
|---|---|---|---|---|---|
| | | | relative | | |
| | | | to | | |
| | Route | | human | Admin. | |
| | of | Dose | clinical | volume | |
| Group | admin | (g/kg) | dose | (ml/kg) | N |
| Blank Control | i.g. | — | — | 10 | 6 |
| Model Control | i.g. | — | — | 10 | 6 |
| Metformin | i.g. | 0.25 | 10 | 10 | 6 |
| Control | | | | | |
| High Dose | i.g. | 5.67 | 21 | 20* | 6 |
| Composition 1 | | | | | |
| Secondary | i.g. | 3.78 | 14 | 10 | 6 |
| High Dose | | | | | |
| Composition 1 | | | | | |
| Middle Dose | i.g. | 2.7 | 10 | 10 | 6 |
| Composition 1 | | | | | |
| Low Dose | i.g. | 1.89 | 7 | 10 | 6 |
| Composition 1 | | | | | |

*The High Dose Composition 1 Group was administered twice a day due to the limited solubility of Composition 1.

### Administration

After the STZ-induced hyperglycemic rat model was successfully established, the rats were randomly divided into different groups except for the Blank Control Group. Each group of animals were given a different dose of Composition 1 or Metformin daily for 30 days. Because of the poor solubility and large dose of Composition 1, the High Dose Group was administrated twice a day for 30 days.

### Determination of Blood Glucose Levels

About 0.5 ml of blood was collected from the posterior orbits vein of rats tested at 72 hours after STZ injection, and then weekly after administration and at the end of administration. Blood samples were centrifuged at 3,500 rpm for 15 min. to obtain serum for determination of the blood glucose level.

### Results

### General Observation

During the administration period, none of the rats died. All animals exhibited good mental state, normal activities and clean fur, and had no piloerection. There were also no other abnormal toxic symptoms to be observed.

### Determination of Blood Glucose Levels

During the entire period of administration, the blood glucose levels of rats in the Model Control Group were significantly higher (p<0.05) than those of the Blank Control Group, indicating that the hyperglycemic rat model induced by STZ was stable and reliable for evaluating anti-hyperglycemic agents.

Comparing to the Model Control Group, the blood glucose levels of rats in the Metformin Control Group were lower (p<0.05) at the 3^{rd} and 4^{th} weeks after the beginning of administration. In the 4 weeks of treatment, the blood glucose levels of hyperglycemic rats in all Composition 1 dose groups tended to decline. Especially for the Middle Dose Group, the blood glucose levels decreased significantly compared to those for the Model Control Group (p<0.05).

Comparing to the blood glucose levels prior to the treatment, the end-point (after the 4-week treatment) blood glucose levels of hyperglycemia rats in the Metformin Control Group decreased by 59%, while for the Composition 1 treatment groups, the end-point blood glucose levels decreased by 20% for the Low Dose Group, 45.5% for the Middle Dose Group, 22% for the Second High Dose Group, and 42% for the High Dose Group.

### Conclusions

The results of this study indicated that Composition 1 tablets at all dose levels (1.89, 2.7, 3.78, and 5.67 g/kg body weight/day) resulted in significant decrease (greater than 20%) in the blood glucose levels of hyperglycemic rats induced by STZ after 4 weeks of treatment. The best time-efficacy responses occurred at the dose level of 2.7 g/kg body weight/day.

### Example 7

### Human Case Studies

Case One: Male, born in 1934. In early 1980's, he was diagnosed with type II diabetes. His highest blood glucose level reached more than 300 mg/dl. He experienced thirst, increased amount and frequency of urine, skin titillation, gum disease, vision decline, joint pains, and lack of energy. His food intake reduced from 1 lb per day to ¼ lb per day. The amount of his medicine for his diabetes had to increase from time to time during these years to bring his blood glucose level down. Even so, the average level of his blood glucose still climbed. In the early 2003, he started to take Composition 1. After 2 weeks, his blood glucose level started dropping. He started to reduce his medicine prescribed for diabetes. After 4 months of reduction, he stopped the prescription and tried to gradually reduce the amount of Composition 1 he was taking. After 2 more months, he stopped taking Composition 1 as well, while his blood glucose level remained around 100 mg/dl. His diet reverted back to normal, his joint pains disappeared, and his energy also reverted back to normal. He is in very good shape and eating candies now.

Case Two: Male, born in 1930. First diagnosed in 1988 with blood glucose levels of 6.2-7.2 mmol/L (fasting) and 11.2-12.5 mmol/L (postprandial). In 2000, he started, to experience skin titillation and vision decline; in 2002, his blood glucose level climbed to 11.6-13 mmol/L (fasting) and 19.7-23.3 mmol/L (postprandial). Major medicines for his diabetes were Metformin (in 1997) and Insulin (28-30 U/d) (in 2002). As a result, his BGL maintained at 5.4-6.5 mmol/L (fasting) and 7.6-9.9 mmol/L (postprandial). In February of 2004, he started to take Composition 1. After two weeks, he started to decrease insulin intake gradually. In about 2 months, he was off insulin completely while maintaining his BGL at 7.2 mmol/L (fasting) and 10.7 mmol/L (postprandial). After continuing to take Composition 1 for another 2 months, his BGL was back to the normal level of 4.4-6.1 mmol/L. Since then, he has been off all medication and living a healthy life-exercising at least 30 min a day.

Case Three: Male, born in 1947, weighing 215 lb. He was first diagnosed with diabetes in 2002. Highest blood glucose level was 166 mg/dl. Major symptoms included thirst, frequent urination, skin titillation, and some neuropathic symptoms. Other medicines used included Ranitidine for acid reflux. He started to take Composition 1 in 2004. Each week he observed his blood glucose level going down. After 2 months, his blood glucose level was back to normal (86 mg/dl).

Case Four Female, born in 1941. She was first diagnosed with diabetes in 2000. Her blood glucose level reached about 250 mg/dl with common symptoms. She started to take Composition 1 in late 2004. After 2 weeks, she started reducing her medicine for diabetes. After 2 months of reduction, she stopped her medicine for diabetes and started reducing Composition 1. After a month of reduction, she stopped Composition 1 as well while her blood glucose level went back to normal and all symptoms disappeared.

(Note: 1 mmol/L=18 mg/dl, e.g. 10 mmol/L=180 mg/dl)

### REFERENCES

1. [symbol] Good Laboratory Practice [symbol] . Chinese State Food and Drug Administration. Beijing, August 2003.
2. Guiding Principles for Laboratory Research of New Drugs. Bureau of Drug Administration & Policy of Ministry of Public Health of P.R. China. Beijing, 1993.

All publications cited herein are incorporated by reference in their entirety.

1. A composition comprising Radix Ginseng, Rhizoma Atractylodis Macrocephalae, Radix Glycyrrhizae, Ginger, Tartarian Buckwheat, Rhizoma Phragmitis (Phragmites communis), and Radix Trichosanthis.

2. (canceled)

3. (canceled)

4. The composition of claim 1, wherein Radix Ginseng, Rhizoma Atractylodis Macrocephalae, Radix Glycyrrhizae, Ginger, Tartarian Buckwheat, Rhizoma Phragmitis (Phragmites communis), or Radix Trichosanthis is in the form of superfine powder or extract powder.

5. The composition of claim 4, wherein a half of the composition by weight is in the form of superfine powder and the other half of the composition by weight is in the form of extract powder.

6. The composition of claim 4, wherein the composition is in the form of a tablet or capsule.

7. The composition of claim 6, wherein the composition is in the form of a tablet and the tablet weighs 1.5 g.

8. The composition of claim 7, wherein the total amount of Radix Ginseng, Rhizoma Atractylodis Macrocephalae, Radix Glycyrrhizae, Ginger, Tartarian Buckwheat, Rhizoma Phragmitis (Phragmites communis), and Radix Trichosanthis is 1.2 g.

9. The composition of claim 1, wherein the amount of Radix Ginseng is in the range of 1-50% of the composition by weight, the amount of Rhizoma Atractylodis Macrocephalae is in the range of 5-50% of the composition by weight, the amount of Radix Glycyrrhizae is in the range of 1-20% of the composition by weight, and the amount of Ginger is in the range of 5-30% of the composition by weight; or wherein the total amount of Radix Ginseng, Rhizoma Atractylodis Macrocephalae, Radix Glycyrrhizae, and Ginger is in the range of 30-90% of the composition by weight, and the total amount of Tartarian Buckwheat, Rhizoma Phragmitis (Phragmites communis), and Radix Trichosanthis is in the range of 10-70% of the composition by weight.

10. (canceled)

11. A method of preparing a composition, comprising admixing Radix Ginseng, Rhizoma Atractylodis Macrocephalae, Radix Glycyrrhizae, Ginger, Tartarian Buckwheat, Rhizoma Phragmitis (Phragmites communis), and Radix Trichosanthis to form a composition.

12. The method of claim 11, wherein Radix Ginseng, Rhizoma Atractylodis Macrocephalae, Radix Glycyrrhizae, Ginger, Tartarian Buckwheat, Rhizoma Phragmitis (Phragmites communis), or Radix Trichosanthis is in the form of superfine powder or extract powder.

13. The method of claim 12, wherein the size of the superfine powder is greater than 300 mesh.

14. The method of claim 12, wherein the extract is prepared by extracting slices of Radix Ginseng, Rhizoma Atractylodis Macrocephalae, Radix Glycyrrhizae, Ginger, Tartarian Buckwheat, Rhizoma Phragmitis (Phragr[alpha]ites communis), or Radix Trichosanthis with 40-85% ethanol in water.

15. The method of claim 14, further comprising:
allowing ethanol to evaporate;
adsorbing the remaining aqueous solution to macroporous adsorbent resins;
desorbing the extract from the resins; and
collecting the extract.

16. The method of claim 15, wherein the extract is desorbed from the resins by desorbing the extract in 60-80% ethanol in water and removing the resins, and the extract is collected by allowing ethanol to evaporate and spraying-dry the extract to form powder.

17. A composition prepared according to the method of claim 14.

18. A composition prepared according to the method of claim 15.

19. A composition prepared according to the method of claim 16.

20. (canceled)

21. A method of reducing the blood glucose level in a subject, comprising administering to a subject in need thereof an effective amount of the composition of claim 1.

22. The method of claim 21, wherein the composition is administered orally or intragastrically.

23. The method of claim 21, wherein the subject suffers from diabetes.

24. The method of claim 23, wherein the subject is a human.

25. A method of reducing the blood glucose level in a human diabetic patient, comprising orally administering to a human diabetic patient in need thereof an effective amount of the composition of claim 8, wherein the composition is administered to the human diabetic patient at a daily dose of 10-15 tablets.

26. (canceled)
